(19) United States
(12) Patent Application Publication
(10) Pub. No.: US 2022/0211426 A1
(43) Pub. Date: Jul. 7, 2022

(54) CATHETERS THAT DELIVER PULSED ELECTRICAL FIELD FOR TARGETED CELLULAR ABLATION

(71) Applicant: Mayo Foundation for Medical Education and Research, Rochester, MN (US)

(72) Inventor: Rahmi Oklu, Scottsdale, AZ (US)

(21) Appl. No.: 17/611,723

(22) PCT Filed: May 15, 2020
(86) PCT No.: PCT/US2020/033073

§ 371 Date: Nov. 16, 2021

Related U.S. Application Data
(60) Provisional application No. 62/849,523, filed on May 17, 2019.

(51) Int. Cl. A61B 18/14

(52) U.S. Cl. CPC A61B 18/1492; A61B 2018/00494; A61B 2018/144

(57) ABSTRACT

Devices can be used to deliver pulsed electrical field for targeted cellular ablation. For example, this document describes catheter-based devices that deliver non-thermal irreversible electroporation to treat deep vein thrombosis by ablating cells of the venous thrombus to prevent cellular mechanisms that lead to clot organization, leaving the clot susceptible to physiological degradation.

### CROSS-REFERENCE TO RELATED APPLICATIONS

This application claims the benefit of U.S. Provisional Application Ser. No. 62/849,523, filed May 17, 2019. The disclosure of the prior application is considered part of (and is incorporated by reference in) the disclosure of this application.

### BACKGROUND

### 1. Technical Field

This document relates to devices that deliver a pulsed electrical field for targeted cellular ablation. For example, this document relates to catheter-based devices that deliver non-thermal irreversible electroporation to treat deep vein thrombosis by ablating cells of the venous thrombus to prevent cellular mechanisms that lead to clot organization, leaving the clot susceptible to physiological degradation. Other applications of the catheter include ablation of ductal tissue to treat obstruction in bile ducts and in ureters due to inflammatory conditions or cancer. In these cases, the bile ducts are accessed using ultrasound guidance and a needle for percutaneous transhepatic cholangiography; once the stenotic segments are identified, the catheter is delivered for therapy. For ureter treatment, the kidney urinary collecting system is accessed using ultrasound guidance and a needle and a nephrostogram is performed to identify the stenotic segments; the catheter is subsequently delivered to this area and the therapy is delivered. Another application involves a larger version of the catheter delivered to the stomach either via percutaneous access or from an oropharyngeal access into stomach; this catheter in the stomach can then be used to ablate the gastric mucosa to cause weight loss.

### 2. Background Information

Venous thrombosis is a blood clot that forms within a vein. The center of the clot can begin to create dense connective tissue, making it difficult for the body to break down the clot. Venous thrombosis, particularly deep vein thrombosis (DVT), is a tremendous healthcare burden in the United States, both clinically and financially. DVT is among the most prevalent medical problems today with an estimated annual incidence of approximately 1 million cases, affecting up to 5% of the population during their lifetime.

Once DVT is diagnosed, it is often treated by anticoagulation therapy, which only prevents the progression of the thrombus. Fibrotic venous thrombi can clinically manifest as post-thrombotic syndrome (PTS). Up to 60% of DVT patients develop PTS, which increases the risk for DVT recurrence, often necessitating life-long anti-coagulation, and can severely impact the quality of life, causing chronic venous insufficiency and, at end stage, venous ulcers.

### SUMMARY

This document describes devices that deliver a pulsed electrical field for targeted cellular ablation. For example, this document describes catheter-based devices that deliver non-thermal irreversible electroporation to treat deep vein thrombosis by ablating cells of the venous thrombus to prevent cellular mechanisms that lead to clot organization, leaving the clot susceptible to physiological degradation.

In one aspect, this disclosure is directed to a device for treating a thrombosis. The device can include a sheath, a handle at a proximal end portion of the sheath, a central wire slidably disposed within the sheath, and a basket including a plurality of elongate electrodes surrounding the central wire at a distal end portion of the sheath, where a distal end of each electrode is attached to the central wire. In some cases, the plurality of elongate electrodes can each have a length of about 3 cm to about 7 cm. In some cases, the plurality of elongate electrodes can include six elongate electrodes. In some cases, the six elongate electrodes can create a hexagonal shape around the central wire. In some cases, the basket can have a diameter of about 3 mm to about 10 mm. In some cases, the basket can be configured to expand and collapse via movement of the central wire. In some cases, the basket can be configured to have a plurality of expanded configurations with different diameters. In some cases, the plurality of expanded configurations can differ by about 1 mm. In some cases, the handle can include a plurality of wire ends of the plurality of elongate electrodes, and the plurality of wire ends can be configured to connect to a stimulation device.

In some cases, the basket can be configured to deliver a stimulation therapy to the thrombosis from the stimulation device. In some cases, the stimulation therapy can be provided between an uninsulated portion of the central wire and one or more of the plurality of elongate electrodes. In some cases, the stimulation therapy can be provided between a first elongate electrode of the plurality of elongate electrodes and an adjacent elongate electrode of the plurality of elongate electrodes. In some cases, the stimulation therapy can be provided between a first elongate electrode of the plurality of elongate electrodes and an opposite elongate electrode of the plurality of elongate electrodes.

In another aspect, this disclosure is directed to a device for delivering thermal or non-thermal ablation. The device can include a sheath, a handle at a proximal end portion of the sheath, a balloon at a distal end portion of the sheath, and a plurality of elongate electrodes surrounding the balloon.

In some cases, the plurality of elongate electrodes can each have a length of about 3 cm to about 7 cm. In some cases, the plurality of elongate electrodes can include six elongate electrodes. In some cases, the six elongate electrodes can create a hexagonal shape around the balloon. In some cases, the plurality of elongate electrodes can be configured to expand and collapse as the balloon inflates and deflates. In some cases, the balloon can be configured to inflate to a plurality of expanded configurations with different diameters. In some cases, the plurality of expanded configurations can differ by about 1 mm.

In some cases, the handle can include a plurality of wire ends of the plurality of elongate electrodes, and the plurality of wire ends can be configured to connect to a stimulation device. In some cases, the plurality of elongate electrodes can be configured to deliver a stimulation therapy from the stimulation device. In some cases, the stimulation therapy can be provided between a first elongate electrode of the plurality of elongate electrodes and an adjacent elongate electrode of the plurality of elongate electrodes.

In another aspect, this disclosure is directed to methods of treating or preventing obesity in an subject in need thereof. The methods include providing the device of the disclosure; and delivering a stimulation therapy, generated by the device, to a gastric mucosa of the subject. In some cases, the stimulation therapy is non-thermal irreversible electroporation. In some cases, the stimulation therapy comprises a treatment pulse lasting about 30 microseconds (μsec) to about 100 μsec. In some cases, the stimulation therapy comprises a treatment pulse lasting about 60 microseconds. In some cases, the stimulation therapy comprises about 10 to about 200 treatment pulses.

In some cases, the stimulation therapy comprises about 99 treatment pulses. In some cases, the stimulation therapy comprises a treatment pulse having a square wave. In some cases, the stimulation therapy comprises a frequency of about 0.5 Hertz (Hz) to about 5 Hz. In some cases, the stimulation therapy comprises a frequency of about 1 Hz. In some cases, the stimulation therapy comprises a voltage of about 20 volts per millimeter (V/mm) to about 200 V/mm. In some cases, the stimulation therapy comprises a voltage of about 120 V/mm. In some cases, the subject has type 2 diabetes, metabolic syndrome, insulin resistance, hyperglycemia, dyslipidemia, hypertension, hyperinsulinemia, cardiovascular disease, or any combination thereof. Particular embodiments of the subject matter described in this document can be implemented to realize one or more of the following advantages. In some embodiments, treatment can include pulsed, non-thermal, low voltage electrical fields, such as irreversible electroporation, to ablate the cells of the venous thrombus using the devices provided herein to prevent cellular mechanisms that lead to clot organization in vessels, leaving the clot susceptible to physiological degradation. In some cases, the treatment can cause a drastic decrease in clot fibrosis in about 1 day to about 10 days after the application of external electrical fields in a venous thrombosis.

Another advantage is the pulsed, non-thermal irreversible electroporation can prevent clot fibrosis in a minimally invasive manner. Current irreversible ablation techniques are based on the use of a rigid 18-gauge needle that require accurate image-guided placement to the site of administration, where up to 3000 V/cm is delivered. As such, an advantage of the device provided herein is precise, time-saving, intra-vascular delivery of finely tunable irreversible electroporation to venous thrombi. In addition, the device provided herein permits variable treatment zones that can span the length of the catheter (e.g., up to 90 cm). In contrast, current technology only allows ablation of tissue in increments of 1 cm and would require numerous needle punctures into veins.

In addition to treating DVT, the device can be used for the treatment of catheter infection, chronic thrombosis, venous/arterial neointimal hyperplasia and ductal hyperplasia; it can also be used to ablate gastric mucosa to induce weight loss. Catheter infection could be treated in several approached. For instance, the electrodes could be embedded with the catheter material itself. Other applications of the catheter include ablation of ductal tissue to treat obstruction in bile ducts and in ureters due to inflammatory conditions or cancer. In these cases, the bile ducts are accessed using ultrasound guidance and a needle for percutaneous transhepatic cholangiography; once the stenotic segments are identified, the catheter is delivered for therapy. For ureter treatment, the kidney urinary collecting system is accessed using ultrasound guidance and a needle and a nephrostogram is performed to identify the stenotic segments; the catheter is subsequently delivered to this area and the therapy is delivered. Another application involves a larger version of the catheter delivered to the stomach either via percutaneous access or from an oropharyngeal access into stomach; this catheter in the stomach can then be used to ablate the gastric mucosa to cause weight loss.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is a side view of an example device for delivering targeted cellular ablation in accordance with some embodiments provided herein.

FIG. 2 is a perspective view of the device of FIG. 1.

FIG. 3 is a close-up perspective view of a distal end portion of the device of FIG. 1.

FIG. 4 is a close-up side view of a first example distal end portion of the device of FIG. 1.

FIG. 5 is a close-up side view of a second example distal end portion of the device of FIG. 1.

FIG. 6 is a side view of another example device embodiment for delivering targeted cellular ablation in accordance with some embodiments provided herein.

FIGS. 7A, 7B, 7C, and 7D are graphs assessing the effects of irreversible electroporation (IRE) treatment of the gastric tissue on body weight and fat mass in diet-induced obese mice. FIG. 7A shows change in body weight of IRE treatment and control groups. FIG. 7B shows food intake of IRE treatment and control groups. FIG. 7C is a graph showing NMR measurements of fat mass of IRE treatment and control groups. FIG. 7D shows heat production of IRE treatment and control groups.

FIGS. 8A and 8B are graphs assessing the long term effects of IRE treatment in diet-induced obese mice. FIG. 8A is shows change in body weight of IRE treatment and control groups. FIG. 8B shows change in fat mass of IRE treatment and control groups.

FIGS. 9A, 9B, and 9C are light microscopy images of tissue sections of the stomach wall of obese mice. FIG. 9A is a light microscopy image of a stained tissue section of a murine stomach wall. FIG. 9B is a representative light microscopy image of a ghrelin-immunostained (brown dots) stomach histology section obtained from an obese mouse showing an IRE treated region. FIG. 9C is a representative light microscopy image of a ghrelin-immunostained (brown dots) stomach histology section obtained from an obese mouse showing an untreated region.

FIGS. 10A, 10B, 10C, and 10D are graphs showing the effect of IRE treatment on metabolic hormones in diet induced obese mice following IRE treatment. FIG. 10A shows ghrelin protein levels in arbitrary units (AU) in IRE treatment and control groups. FIG. 10B shows leptin levels in IRE treatment and control groups. FIG. 10C is shows amylin levels in IRE treatment and control groups. FIG. 10D shows peptide YY (PYY) levels in IRE treatment and control groups.

Like reference numbers represent corresponding parts throughout.

### DETAILED DESCRIPTION

This document describes devices that deliver a pulsed electrical field for targeted cellular ablation. For example, this document describes catheter-based devices that deliver non-thermal irreversible electroporation to treat deep vein thrombosis by ablating cells of the venous thrombus to prevent cellular mechanisms that lead to clot organization, leaving the clot susceptible to physiological degradation.

Venous thrombosis, particularly deep vein thrombosis (DVT), is a tremendous burden on the US healthcare, both clinically and financially. DVT is among the most prevalent medical problems today with an estimated annual incidence of approximately 1 million cases, affecting up to 5% of the population during their lifetime.

Once DVT is diagnosed, it is commonly treated by anticoagulation therapy, which only prevents the progression of the thrombus and does not address the intense cellular activity within the thrombus. This intra-thrombus cellular activity mediates the organization and fibrosis of the thrombus. Since there is often incomplete resolution of the DVT despite the best medical therapy, fibrotic changes in the residual thrombus reduces the efficacy of anticoagulation therapy as well as intravenous and catheter directed interventions.

Fibrotic venous thrombi can clinically manifest as post-thrombotic syndrome (PTS). Up to 60% of DVT patients develop PTS, which increases the risk for DVT recurrence often necessitating life-long anti-coagulation and can severely impact the quality of life, causing chronic venous insufficiency and, at end stage, venous ulcers. Thus, there is a great unmet need in medicine today to identify ways in which DVT can be effectively treated, avoiding PTS and its debilitating complications. An ideal treatment should not only address thrombus progression with anticoagulation but also the internal cellular activity that leads to the fibrosis of the blood clot.

In some embodiments, the devices provided herein can deliver treatment that can include pulsed, non-thermal, low voltage electrical fields, such as irreversible electroporation, to ablate the cells of the venous thrombus using the devices provided herein to prevent cellular mechanisms that lead to clot organization in vessels, leaving the clot susceptible to physiological degradation. The devices described herein can provide a pulsed, non-thermal irreversible electroporation that can prevent clot fibrosis in a minimally invasive manner. Another advantage of the devices provided herein is precise, time-saving, intra-vascular delivery of finely tunable irreversible electroporation to venous thrombi. In addition, the device provided herein permits variable treatment zones that can span the length of the catheter (e.g., up to 90 cm). In addition to treating DVT, the device can treat catheter infection, chronic thrombosis, venous/arterial neointimal hyperplasia, renal hypertension and ductal hyperplasia.

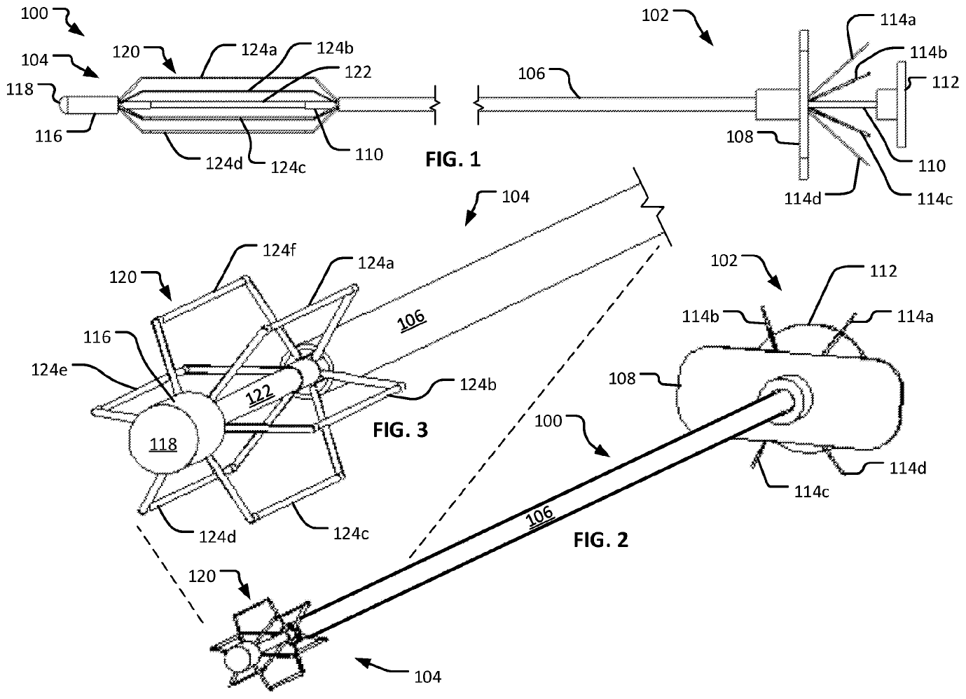

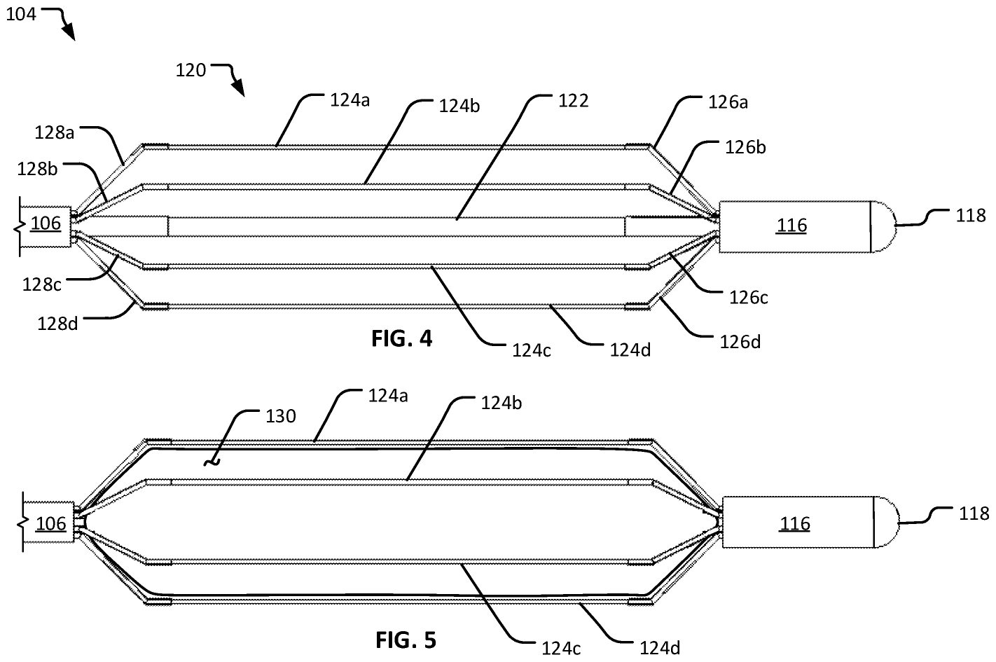

Referring to FIGS. 1-4, a device 100 for delivering targeted cellular ablation can include a proximal portion 102, a distal portion 104, and a sheath 106 extending from proximal portion 102 to distal portion 104. In some cases, sheath 106 can be a standard vasculature sheath.

Proximal portion 102 can include a handle 108, a central wire 110, a push member 112, and wire ends 114a, 114b, 114c, and 114d. Handle 108 can extend from sheath 106 and provide support for fingers of a user. In some cases, handle 108 can extend perpendicular to sheath 106.

Central wire 110 can extend through sheath 106. In some cases, central wire 110 can be insulated. Push member 112 can be coupled to central wire 110 and wire ends 114a, 114b, 114c, and 114d. Wire ends 114a, 114b, 114c, and 114d can be insulated. In some cases, wire ends 114a, 114b, 114c, and 114d can include an uninsulated portion for connection to a stimulation device. In some cases, wire ends 114a, 114b, 114c, and 114d can be connected to a stimulation device in different configurations to provide different stimulation configurations. In some cases, wire ends 114a, 114b, 114c, and 114d can extend from sheath 106 at an outward angle.

Distal portion 104 can include a distal end 116, a tip 118, a basket 120, and an uninsulated wire 122. Distal end 116 can be distal to basket 120. In some cases, distal end 116 has a diameter substantially similar to a diameter of sheath 106. Tip 118 can be the distal-most end of device 100. In some cases, tip 118 can be atraumatic. For example, tip 118 can be blunt. In some cases, tip 118 can be rounded. In some cases, distal end 116 and tip 118 are integral.

Basket 120 can include one or more elongate electrodes 124a, 124b, 124c, 124d, 124e, and 124f Elongate electrodes 124a, 124b, 124c, 124d, 124e, and 124f can be uninsulated, creating a treatment zone. In some cases, elongate electrodes 124a, 124b, 124c, 124d, 124e, and 124f create a hexagonal treatment zone. In some cases, the treatment zone can be a pentagon, an octagon, and so on. In some cases, elongate electrodes 124a, 124b, 124c, 124d, 124e, and 124f can have a length between about 1 cm and about 10 cm. In some cases, elongate electrodes 124a, 124b, 124c, 124d, 124e, and 124f can have a length between about 3 cm and about 7 cm.

In some cases, elongate electrodes 124a, 124b, 124c, 124d, 124e, and 124f can include distal insulated portions 126a, 126b, 126c, 126d, 126e, and 126f, respectively. In some cases, elongate electrodes 124a, 124b, 124c, 124d, 124e, and 124f can include proximal insulated portions 128a, 128b, 128c, 128d, 128e, and 128f, respectively. Elongate electrodes 124a, 124b, 124c, 124d, 124e, and 124f can each include a proximal portion (e.g., wire ends 114a, 114b, 114c, 114d, 114e (not shown), and 114f (not shown), respectively) that extends through the length of sheath 106 and out of a proximal end of sheath 106. In some cases, elongate electrodes 124a, 124b, 124c, 124d, 124e, and 124f can be flexible.

In some cases, basket 120 can be made of a material with shape memory. For example, basket 120 can be made fully or partially of nitinol. Basket 120 can expand and collapse based on a position of push member 112 relative to sheath 106. For example, in a first position of push member 112, basket 120 can be collapsed. In some cases, when basket 120 is collapsed, basket 120 can be fully or partially positioned inside sheath 106. In a second position of push member 112, basket 120 can be partially expanded. In a third position of push member 112, basket 120 can be fully expanded. In some cases, pulling push member 112 away from support 108 causes basket 120 to collapse. In some cases, push member 112 can have a plurality of preset positions that correspond to preset diameters of basket 120. For example, push member 112 can have four preset positions, such as collapsed, fully expanded, and two positions with different diameters where basket 120 is partially expanded. In some cases, a diameter of basket 120 can change by 1 mm when moving between various positions.

The diameter of the device, for example from elongate electrode 124f to elongate electrode 124c or elongate electrode 124b to elongate electrode 124e, can be made to vary by either pushing or pulling the push member 112 or handle 108. The combination of push member 112 and handle 108 can be used to alter the diameter of the electrodes to accommodate the diameter of the vein. For example, with tension applied to push member 112, handle 108 can be pushed in to reduce the diameter of the device. Alternatively, the device could have fixed diameters; computerized tomography (CT), ultrasound or angiography images are used to estimate the diameter of the vein and the appropriate catheter with the corresponding diameter is delivered to the thrombosed segment for treatment. In some cases, the diameter of basket 120 can be about 3 mm to about 10 mm. In some cases, the diameter of basket 120 can correspond to a diameter of a blood vessel in which the device is placed. In some cases, one or more of elongate electrodes 124a, 124b, 124c, 124d, 124e, and 124f touch a wall of the blood vessel. In some cases, basket 120 can expand into a thrombosis.

Uninsulated wire 122 can be an uninsulated portion of central wire 110. In some cases, elongate electrodes 124a, 124b, 124c, 124d, 124e, and 124f can be positioned about 3.5 mm from uninsulated wire 122. In some cases, elongate electrodes 124a, 124b, 124c, 124d, 124e, and 124f can expand between about 1 mm to about 6 mm from uninsulated wire 122.

In some cases, uninsulated wire 122 can be an anode or a cathode, and one or more of elongate electrodes 124a, 124b, 124c, 124d, 124e, and 124f can be the other of an anode or a cathode to provide treatment between uninsulated wire 122 and one or more elongate electrodes 124a, 124b, 124c, 124d, 124e, and 124f. In some cases, one or more of elongate electrodes 124a, 124b, 124c, 124d, 124e, and 124f can be an anode or a cathode, and another one or more elongate electrodes 124a, 124b, 124c, 124d, 124e, and 124f can be the other of an anode or a cathode to provide treatment between multiple elongate electrodes 124a, 124b, 124c, 124d, 124e, and 124f. In some cases, adjacent elongate electrodes 124a, 124b, 124c, 124d, 124e, and 124f can be used to provide treatment. In some cases, opposite elongate electrodes 124a, 124b, 124c, 124d, 124e, and 124f can be used to provide treatment.

In some cases, elongate electrodes 124a, 124b, 124c, 124d, 124e, and 124f and uninsulated wire 122 are tunable such that elongate electrodes 124a, 124b, 124c, 124d, 124e, and 124f and uninsulated wire 122 can be modified to be either an anode or a cathode. In some cases, uninsulated wire 122 and elongate electrodes 124a, 124b, 124c, 124d, 124e, and 124f can be subjectively activated to create treatment zones between uninsulated wire 122 and one or more 124a, 124b, 124c, 124d, 124e, and 124f. In some cases, uninsulated wire 122 and elongate electrodes 124a, 124b, 124c, 124d, 124e, and 124f can be activated in combination with one another to create multiple treatment zones. In some cases, the device can include sensors (e.g., the elongate electrodes 124a, 124b, 124c, 124d, 124e, and 124f). The sensors can measures Amps, creating a feedback loop. If a certain Amp is detected, the catheter can reduce or terminate stimulation to prevent thermal injury. In some cases, the device can include temperature sensors and include a feedback loop system to ensure that thermal injury does not occur to the tissue being ablated.

In some cases, treatment can be electrical field ablation. In some cases, treatment can be irreversible ablation. In some cases, treatment can include low frequency pulses. For example, treatment pulses can last about 30 μseconds to about 100 μseconds. As another example, treatment pulses can last about 60 μseconds to about 70 μseconds. In some cases, pulses can be a square wave. In some cases, treatment can be provided at a frequency of about 0.5 Hz to about 5 Hz. For example, treatment can be provided at a frequency of about 1 Hz to about 2 Hz. In some cases, treatment can be provided for about 30 seconds to about 120 seconds. For example, treatment can be provided for about 60 seconds to about 90 seconds. In some cases, treatment can be delivered with an amplitude to obtain a voltage of about 40 V/mm to about 100 V/mm. For example, treatment can be delivered with an amplitude to obtain a voltage of about 60 V/mm to about 80 V/mm.

Referring to FIG. 5, a second embodiment of distal portion 104 of device 100 can include a balloon 130. Balloon 130 can be positioned within basket 120. In some cases, inflation of balloon 130 can cause elongate electrodes 124a, 124b, 124c, 124d, 124e, and 124f to expand. In some cases, balloon 130 have a diameter such that when balloon 130 is inflated, one or more elongate electrodes 124a, 124b, 124c, 124d, 124e, and 124f abut a wall of a vessel being treated. In some cases, adjacent elongate electrodes 124a, 124b, 124c, 124d, 124e, and/or 124f can be used to deliver an electrical field.

In some cases, device 100 with balloon 130 can be used for denervation (for treatment of hypertension), treatment of a ureter, treatment of bile ducts, and/or treatment of a carcinoma. In some cases, cells of a duct of a patient cause cancer and thermal treatment causes a loss of patency of the duct, which can lead to infections. By using non-thermal ablation delivered via elongate electrodes 124a, 124b, 124c, 124d, 124e, and 124f on an exterior of balloon 130, patency of the duct can be preserved as no thermal damage is done.

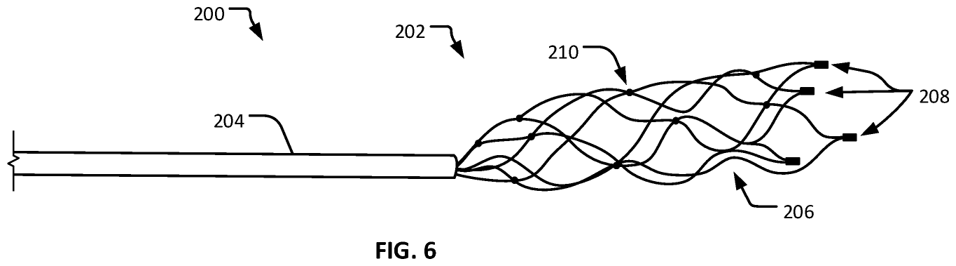

Referring to FIG. 6, a device 200 for delivering targeted cellular ablation can include a distal portion 202 and a catheter 204. Distal portion 202 can include a lattice structure 206 of elongate electrodes. In some cases, lattice structure 206 can be insulated. Lattice structure 206 can include focal electrodes 208 and electrodes 210. Focal electrodes 208 can be located at a distal end portion of lattice structure 206. Electrodes 210 can be located proximal focal electrodes 208. In some cases, electrodes 210 are dispersed throughout lattice structure 206.

In some cases, the devices described herein can be used for a variety of applications. For example, the device can be used in a ureter, a bile duct, a stomach, arteries, or other vessels and ducts of a patient. In some cases, the device can be used for denervation, killing cancer cells, killing bacteria, weight loss, etc. In some cases, the device can be used for treatment of atherosclerosis, hyperplasia, renal hypertension, etc. In some cases, the devices can be used to kill bacteria in another device, such as a catheter. For example, the device can be used to kill bacteria in a dialysis catheter. In some cases, in other applications, a less intense electrical field can be delivered. For example, the electrical field can be applied at about 5 V/mm to about 10 V/mm.

In some cases, the microelectrode arrays (e.g., basket 120, lattice structure 206, etc.) can be created using a mat of ultrathin (50 μm) PE membrane that can be fabricated by spin-casting melted PE at an elevated temperature (>150° C.). Spin-coating speed can be varied to control the thickness of the mat. An array of eight evenly spaced (300 μm) gold microelectrodes with a width of 200 μm can be screen-printed onto a mat with a size of 2.4 by 10 mm2. The printing can be carried out by attaching onto the PE mat a shadow mask made of a transparent tape (thickness: 50 μm) prepared by laser cutting, on which conductive ink can be pasted, desiccated for 1 hour in air, and allowed to completely anneal at 50-70° C. for 2-12 hours.

In some cases, the shadow mask can be gently peeled off to expose the patterned microelectrodes. This printing process can be repeated until a desired thickness of the electrodes is achieved. Finally an insulating ink can be used to define the working electrodes under a curing condition of 20 min at 70° C. The flexible PE mat impregnating microelectrode arrays can then be subjectively placed on each tine of the catheter and two clamps at each side will be used to fix the mat. The clamps can be designed with a wiring system that connects to the external IRE machine from the interior of the catheters. The complex can then be heated for 6 hours at 80-100° C., the glass transition temperature (Tg) of PE (the material of both the mat and the catheter tip), so that the two will seamlessly integrate to permanently fix the electrodes on catheter tines. A 7 French catheter can be used to collapse and unsheathe the device, for example, in animal experiments.

### Experiment 1

A vein model can be fabricated according to a 3-step template micromolding technique based on 3D bioprinting as previously described. A bioprinter equipped with pumps and dispensing capillaries can be used. A capillary size of 900 μm can be chosen to mimic the size of the femoral veins of rats (600-1000 μm).

To form an endothelial monolayer, microchannels can be slowly perfused with a suspension of 0.5-1×107 mL-1 rat primary ECs and incubated for 3 hr. In order to achieve a uniform seeding, the cell perfusion and incubation can be repeated twice and the channels can be flipped upside down in between the two steps. The microchannels seeded with ECs can be maintained in a complete endothelial cell medium for 3-10 days until a tight layer of cells is observed.

Freshly drawn rat whole blood can be mixed with 5 μM Calcein Blue and Sytox Orange into the endothelialized microchannels in the hydrogel block. The endothelialized microchannels filled with blood can be left for 10 min until the thrombus was stabilized. As a control, into the microchannel 10% (v/v) 0.1 M CaCl2 solution in PBS can be injected via a 32G needle to expedite coagulation and the formation of the artificial thrombus in the vein model. The thrombosed vein model can then be maintained in the complete endothelial cell medium for up to 7 days to allow for aging and fibrosis of the thrombus.

The fabricated channels can be perfused with PBS containing dye at flow rates of 50-1000 μL h-1 to visually examine their connectivity. The endothelium within these channels can be characterized by immunostaining for CD31 and tight junction protein ZO-1 as well as DAPI for nuclei.

Transition from live to dead cells during irreversible electroporation (IRE) treatment can be monitored and quantitated in real time using a LIVE/DEAD viability assay in time-lapse fluorescent microscopy where Calcein Blue-stains live cells and Sytox Orange stains dead cells. The samples can also be subjected to histology analysis with hematoxylin-eosin (H&E) staining at regions where IRE is applied. Temperature change during IRE treatment can be monitored to ensure non-thermal conditions by inserting a thin probe (IT-21, ∅=0.4 mm) into the proximity of the electrodes through the gel followed by USB-TC01 Thermocouple Measurement Device.

By varying the seeding density and culture period of the cells, these parameters can be optimized for formation of a complete monolayer of rat ECs. ECs >95% positive for ZO-1 staining were considered effective monolayers. Systematic study on non-thermal IRE treatment can lay out the fundamental understanding of the optimal conditions under which the ECs and nucleated blood cells can be eliminated. Parameters that result in >90% non-thermal (<38° C.) ablation of blood cells and ECs with IRE treatment can be used for rational design of flexible electrodes on catheters. Using ImageJ, fluorescence images in blue (calcein) and orange (Sytox) channels can be used in measuring percent ablation score before and after IRE at 10 min. The preliminary data using PDMS chips indicated >98% ablation at 7 min post IRE treatment using 80 V/mm at a 70 μs duration and 1 Hz frequency for 99 pulses.

In some cases, real time monitoring of live-to-dead transition of cells during IRE treatment in the presence of human whole blood may present an obstacle due to the opacity of the blood. If this is the case, IRE can be applied first, then the treated segments can be sliced longitudinally using a razor blade and then fluorescent imaging can be performed on at least 10 random slices.

Using the 3D bioprinted thrombosed vein model, the capability can be verified and the parameters of non-thermal IRE generated by the microelectrode arrays integrated onto the catheter for efficient elimination of cells can be optimized. The catheter can be slowly inserted into the vessel and unsheathed; either subject electrode pairs can then be activated creating an electric field to induce IRE, or all pairs can be activated simultaneously.

A subset of electric field density, pulse duration, and application cycles can be used; electric field density (40, 80, 100, 160 V mm-1), pulse duration (40, 60, 80 μs), and application cycles (50, 100, 200) can be systematically evaluated to identify key factors on the elimination of rat ECs and nucleated cells in the blood clot. Frequency can be maintained at 1 Hz. ECM 830 Square Wave Electroporation System can be used for signal generation. These electrical parameters can be evaluated to fine-tune the IRE treatment conditions using LIVE/DEAD cell assay. The thickness of the microelectrodes can also be adjusted to ensure good contact with the blood clot while maximizing their stability against the catheter surface.

Morphology and adhesion of the microelectrode arrays can be characterized by scanning electron microscopy (SEM). Stability of the microelectrodes can be evaluated through impedance measurement under shear flow at physiological values (5-20 dyne cm-2 for veins and venules) in rat whole blood and by repeated insertion into the clotted hydrogel microchannels for 50-100 cycles. Cell viability and temperature change before/during/after IRE can be performed as described above. Microelectrodes fabricated under conditions that maintain their adhesion and impedance within 10% under shear analysis can be deemed stable. Parameters that result in >90% non-thermal (<38° C.) ablation of nucleated thrombus cells and ECs with IRE treatment can be used for additional experiments described below.

The adhesion of the PE mat may not be strong enough to hold the printed microelectrode arrays under mechanical stimulation. If this occurs, surface treatment such as brief etching using oxygen plasma can be used to increase the surface roughness of the mat and therefore the adhesion to the printed electrodes. In some cases, an electrodeposition method can be used to fabricate the electrodes by successive deposition of titanium and gold layers for improved adhesion.

### Experiment 2

A clinically relevant in vivo animal model can be used for evaluating the translational potential of the device described herein. A rat femoral vein thrombosis model recapitulates the fibrosis and organization seen in a human DVT. Preliminary studies have demonstrated that, by externally applying IRE on surgically exposed femoral veins, organization was prevented of 7-day-old DVTs, enabling their natural breakdown. In the untreated 7-day-old control veins there is severely fibrotic thrombus. At 7 days the vein patency by histology is >90% in the treated veins and <10% in non-treated veins (n=5 rats). In post-IRE 3-day-old clots only a few nucleated cells are seen, demonstrating the efficient killing of non-thermal IRE, in marked contrast to the cell-laden DVT in the control vein. Immunohistochemistry revealed minimal, if any, neutrophil extracellular traps (NETS) released by ablated neutrophils in the IRE treated veins. Western blotting also demonstrated reduced secretion of tissue factor and reduced transforming growth factor-beta, which is associated with fibrosis.

A pig iliac vein endovascular thrombosis model can be created in several pigs (e.g., 15 pigs). Briefly, the femoral vein can be accessed using an 18G needle and a 0.035 inch Bentson wire; over this wire a 7 French×11 cm sheath can be placed. Over this Bentson wire, a 5 French catheter can be placed at the proximal common iliac vein where an Amplatzer Plug can be deployed or surgically tied using a suture to induce thrombosis in the distal iliac vein. Approximately 2 hours after the formation of venous thrombus, the DVT-IRE catheter can be delivered to the thrombosed iliac vein and treated using parameters optimized in the rat experiments. The untreated thrombosed internal iliac vein samples served as controls.

Following intervention, the sheaths, catheters and wires can be removed and hemostasis can be achieved by manual compression. The animals can be recovered from anesthesia and observed for two weeks.

Animals can be sacrificed 14 days after the model creation for histologic analysis. H&E, Masson's trichrome, Martius Scarlet Blue (to quantitate fibrin) and reticulin staining (to assess thermal injury); immunostaining for collagen, neutrophils, NETS, macrophages, platelets, endothelium, smooth muscle cells and tissue factor can be performed. Using ImageJ, vein patency and vein circumference (to exclude aneurysms) can be measured; immunostaining signal can be quantitated and nucleated cell numbers in tissue sections can be counted. Cytokine arrays can also be fully analyzed comparing protein extracts from ablated thrombi versus controls.

The application of intra-vascular non-thermal IRE via a microelectrode array integrated on catheters can provide highly efficient ablation of cells in the porcine DVT to prevent clot fibrosis. Patency can be calculated based on histology images and gross evaluation; IRE treatment resulting in >80% patency can be considered effective de-clotting. Parameters should also induce >90% ablation of nucleated cells without thermal injury. Untreated veins will demonstrate significant gross fibrosis of the DVT. In an aspect, this disclosure describes methods of treating obesity in a subject (e.g., a mammal, e.g., a human or non-human veterinary subject, e.g., a dog, cat, horse, primate, rodent, or pig) in need thereof. The methods can include providing the device of the disclosure and delivering a stimulation therapy, generated by the device, to a gastric mucosa of the subject. In some embodiments, the stimulation therapy is non-thermal irreversible electroporation. The stimulation therapy can include treatment pulses with parameters (e.g., duration, wave type, frequency, and amplitude to obtain a voltage) as described elsewhere herein.

In some embodiments, subjects can be obese subjects, overweight subjects, subjects with a higher than normal Body Mass Index (BMI), subjects having type 2 diabetes, subjects having metabolic syndrome, subjects having insulin resistance, subjects having hyperglycemia, subjects having dyslipidemia, subjects having hypertension, subjects having hyperinsulinemia, and/or subjects having cardiovascular disease. In some embodiments, subjects having dyslipidemia have decreased high density lipoprotein (HDL) levels, elevated low density lipoprotein (LDL) levels, and/or elevated triglycerides. In some embodiments, the devices of the disclosure can be used to promote weight loss and/or reduce body fat percentage of a body composition of the subject. In some embodiments, the device can be used to treat, prevent, and/or reduce type 2 diabetes, metabolic syndrome, insulin resistance, hyperglycemia, dyslipidemia, hypertension, hyperinsulinemia, and/or cardiovascular disease.

Ghrelin is a circulating hormone produced mainly by enteroendocrine cells of the gastrointestinal tract, especially the stomach, and is often called a “hunger hormone” because it stimulates appetite, increases food intake, and promotes fat storage. Thus, it may be desirable to decrease ghrelin levels in subjects in need thereof. In some embodiments, IRE treatment with the devices disclosed herein decrease, prevent an increase of, or inhibit ghrelin in subjects. As used herein, the expressions “inhibition of ghrelin” or “inhibit ghrelin” refer to an impairment of the biological activity of ghrelin, which occurs due to a decrease in ghrelin levels and/or due to an impairment of its biological activity.

Leptin is a hormone released by adipocytes that helps regulate and alter long-term food intake and energy expenditure. Therefore, subjects exhibiting a decrease in fat mass may show decreases leptin levels. In some embodiments, IRE treatment with the devices disclosed herein decrease, prevent an increase of, or inhibit leptin in subjects in need thereof (relative to untreated subjects) as a result of a decreased body fat percentage. As used herein, the expressions “inhibition of leptin” or “inhibit leptin” refer to an impairment of the biological activity of leptin, which occurs due to a decrease in leptin levels and/or due to an impairment of its biological activity. Amylin is a hormone that is co-secreted with insulin from pancreatic beta-cells. Amylin helps regulate blood glucose levels by slowing gastric emptying and promoting satiety, thereby preventing post-prandial spikes in blood glucose levels. Its overall effect is to slow the rate of appearance of glucose in the bloodstream after eating. Thus, it may be desirable to increase amylin levels in subjects in need thereof.

In some embodiments, IRE treatment with the devices disclosed herein increase, prevent a decrease of, stimulate production of, or activate amylin in subjects in need thereof relative to untreated subjects. In some embodiments, IRE treatment with the devices disclosed herein activate amylin in subjects in need thereof. As used herein, the expressions “activation of amylin” or “activate amylin” refer to an enhancement of the biological activity of amylin, which occurs due to an increase in amylin levels and/or due to an enhancement of its biological activity.

Peptide YY (PPY), also known as peptide tyrosine tyrosine, is secreted by cells in the ileum and colon in response to feeding and has been shown to reduce appetite. Thus, it may be desirable to increase PPY levels in subjects in need thereof. In some embodiments, IRE treatment with the devices disclosed herein increase, prevent a decrease of, stimulate production of, or activate PPY in subjects in need thereof relative to untreated subjects. In some embodiments, IRE treatment with the devices disclosed herein activate amylin in subjects in need thereof. As used herein, the expressions “activation of PPY” or “activate PPY” refer to an enhancement of the biological activity of PPY, which occurs due to an increase in PPY levels and/or due to an enhancement of its biological activity.

### EXAMPLES

### Example 1—Assessment of Irreversible Electroporation (IRE) Treatment in a Mouse Model

A mouse model of diet-induced obesity was used to test the translational potential of the device described herein to induce weight loss, reduced food intake, and lower fat mass. Diet-induced obese (DIO) C57-BL/6 mice were maintained on high fat diet (60% kcal was derived from fat) starting at 5 weeks until they reached an average age of 19 weeks to simulate the metabolic syndrome in human. All mice were subjected to mid-abdomen incision and exposure of the stomach; then, they were randomly divided into IRE or control groups.

To test the device of the disclosure in mice, animals were anesthetized with isoflurane inhalation in 100% oxygen and placed in a supine position on a warming platform to maintain the central temperature at 37° C. The abdominal hair was removed and the skin was wiped with betadine solution and the abdomen was surrounded with sterile drapes. A midline abdominal incision was made to expose the abdominal muscles which was cut along the avascular linea alba. The fat tissue was pushed aside with sterile cotton swabs and the abdominal wall was retracted. The device of the disclosure was inserted inside the stomach through a small incision made in the greater curvature of the squamous fore stomach. The device of the disclosure was applied to the stomach to span the glandular region of the murine stomach. The device was connected to an electroporation system. The IRE group received a treatment comprising 99 pulses of 120 volts/millimeter (V/mm) at 1 Hz with 60 micro seconds duration delivered to glandular gastric wall. The control sham group received the device but not the IRE treatment (i.e., the device was placed in contact with the glandular gastric wall, but did not deliver treatment pulses). Whole body weight, food intake, and body mass composition were serially assessed for up to five weeks after surgery. Additionally, metabolic studies aimed at assessing caloric expenditure were also performed.

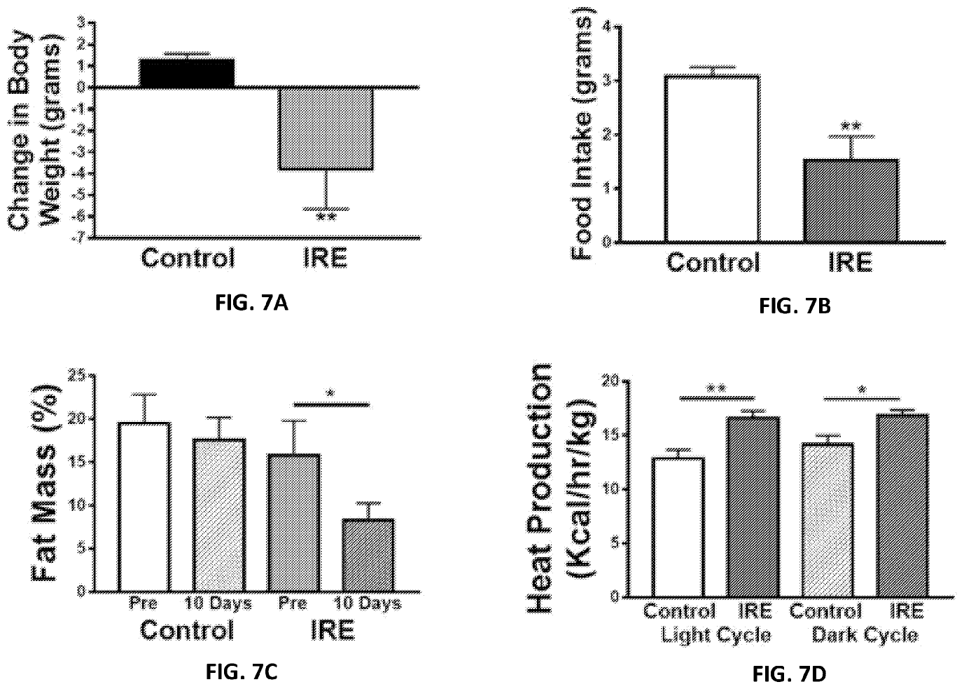

Weight loss was observed in the IRE-treated, diet-induced obese mice compared to the untreated control group over a period of 10 days. During this time period, both groups were placed on a high-fat diet, which resulted in significant weight loss. For example, FIG. 7A is a graphic summary of the change in body weight of IRE-treated mice compared untreated mice in the control group showing that the IRE treatment significantly reduced mice weight compared to control at 10 days after treatment. As shown in FIG. 7A, the change in weight was −6±2.4 grams for the IRE treatment group compared to 2±1.1 grams for the control group (p<0.01). The weight loss was associated with significantly reduced food intake over the 10 day period of the IRE-treated mice compared to the control group, as shown in FIG. 7B (p<0.01). Local ablation of mucosal cells was achieved at levels exceeding >98% at Day 3 post-IRE treatment without significant injury to the submucosa layers of stomach or causing atrophy of the muscularis layers.

More importantly, after 10 days on a high fat diet, mice treated with IRE lost weight compared to controls as corroborated by nuclear magnetic resonance (NMR) analysis of body composition of both IRE-treated and control groups. FIG. 7C is a graph summarizing NMR measurements of fat mass at the end of the 10 day period. As shown in FIG. 7C, the fat mass in the IRE-treated group was reduced compared to the control group (p<0.05). Furthermore, metabolic studies using the Comprehensive Lab Animal Monitoring System documented an increase in calories expenditure in the form of heat production, as shown in FIG. 7D. Increased heat production observed in the IRE-treated mice indicated an altered energy metabolism. FIG. 7D shows an increase heat production in the IRE-treated mice suggesting enhanced caloric expenditure in the animals through heat dissipation during the day (light) and night (dark) periods. Data are mean ±SEM, n=8 in each group (*p<0.05, **p<0.01).

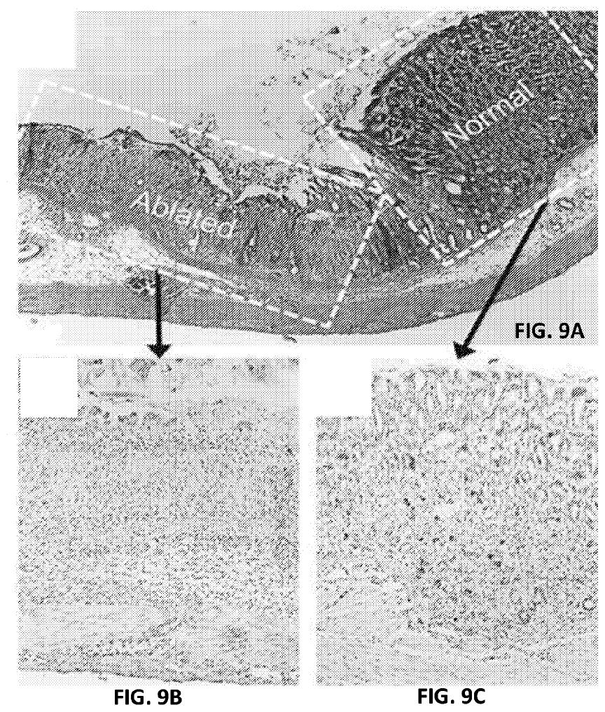

Moreover, as shown in FIGS. 9A-9C, histologic evaluation of tissue section of the stomach walls of the IRE-treated obese mice at 10 days showed significantly less enteroendocrine cells (e.g., ghrelin-expressing cells) in the glandular stomach mucosa and the muscularis layer was preserved. FIG. 9A is a light microscopy image of a hematoxylin and eosin (H&E)-stained tissue section of the stomach wall demonstrating the border zone between IRE-ablated and normal mucosa. FIG. 9B is a representative light microscopy image of a ghrelin-immunostained (brown dots) stomach histology section obtained from an obese mouse showing an IRE-treated region. FIG. 9C is a representative light microscopy image of a ghrelin-immunostained (brown dots) stomach histology section obtained from an obese mouse showing an untreated region. The ghrelin expressing cells are absent from the IRE-treated region compared to the untreated normal region demonstrating effective ablation of the enteroendocrine (e.g., ghrelin-expressing cells) cells.

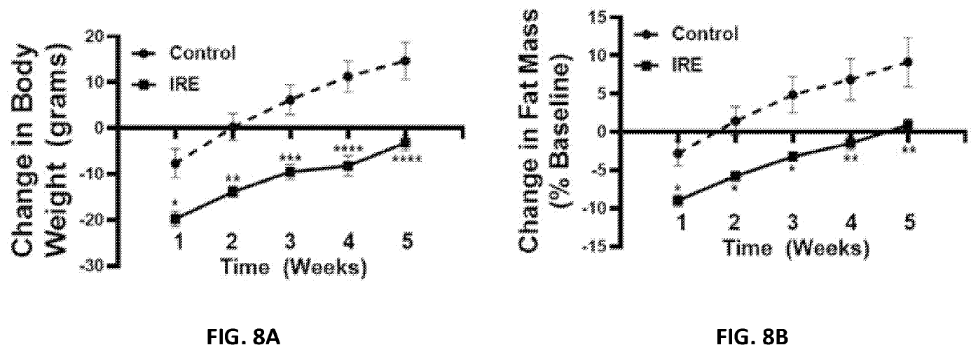

Additional experiments were performed using the same parameters to assess and monitor the durability of the IRE treatment effects on body weight and fat mass in diet-induced obese mice over a prolonged time period. As shown in FIGS. 8A and 8B, these experiments showed that the treatment of the stomach in obese mice using the device of the disclosure led to prolonged weight loss that persisted for up to 5 weeks after treatment. FIG. 8A is a graph summarizing the change in body weight of IRE-treated mice compared to untreated mice in the control group. The body weight data showed that IRE treatment significantly reduced mice body weight compared to the control group throughout the 5 week period. FIG. 8B is a graph comparing NMR measurements of fat mass in control and IRE treatment groups over five weeks. The data demonstrated persistently lower fat mass percentages in IRE-treated mice compared to the control group, which paralleled the reduced body weight data shown in FIG. 8A. Data are mean ±SEM, n=8 in each group (*p<0.05, **p<0.01, ***p<0.001, ****p<0.0001). These experiments demonstrated that non-thermal electric field application to the glandular region of murine stomachs using the device described in this disclosure can lead to weight loss and a decrease in fat mass.

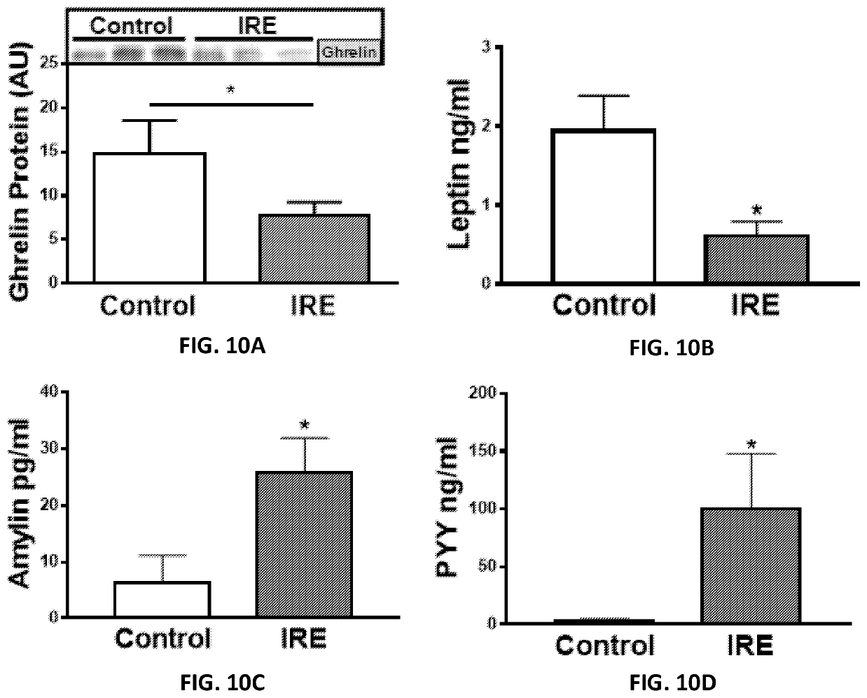

Furthermore, additional experiments were performed to assess the effect of IRE treatment on metabolic hormones in diet-induced obese mice following IRE treatment. Serum and gastric tissue samples were obtained from diet-induced obese mice at 10 days after IRE treatment or sham control procedure. The serum and gastric samples were analyzed for various appetitive regulating biomarkers including ghrelin, amylin, leptin, and peptide YY (PYY), as shown in FIGS. 10A, 10B, 10C, and 10D, respectively. FIGS. 10A and 10B show a significant decrease of ghrelin levels in the stomach wall associated with a decrease in leptin in the serum (*<0.05). Furthermore, FIGS. 10C and 10D show a marked increase in amylin and PYY levels (*<0.05). While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products. Particular embodiments of the subject matter have been described. Other embodiments are within the scope of the following claims.

For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

1. A device for treating a thrombosis, the device comprising:
a sheath;
a handle at a proximal end portion of the sheath;
a central wire slidably disposed within the sheath; and
a basket comprising a plurality of elongate electrodes surrounding the central wire at a distal end portion of the sheath, wherein a distal end of each electrode is attached to the central wire.

2. (canceled)

3. The device of claim 1, wherein the plurality of elongate electrodes comprises six elongate electrodes, each elongate electrode having a length of about 3 cm to about 7 cm.

4. The device of claim 3, wherein the six elongate electrodes create a hexagonal shape around the central wire.

5. The device of claim 1, wherein the basket has a diameter of about 3 mm to about 10 mm.

6. The device of claim 1, wherein the basket is configured to expand and collapse via movement of the central wire, and wherein the basket is configured to have a plurality of expanded configurations with different diameters.

7. (canceled)

8. The device of claim 6, wherein the plurality of expanded configurations differ by about 1 mm.

9. The device of claim 1, wherein the handle includes a plurality of wire ends of
the plurality of elongate electrodes, and wherein the plurality of wire ends are configured to connect to a stimulation device.

10. The device of claim 9, wherein the basket is configured to deliver a stimulation therapy to the thrombosis from the stimulation device.

11. The device of claim 10, wherein the stimulation therapy is provided between an uninsulated portion of the central wire and one or more of the plurality of elongate electrodes.

12. The device of claim 10, wherein the stimulation therapy is provided between a first elongate electrode of the plurality of elongate electrodes and an adjacent elongate electrode of the plurality of elongate electrodes.

13. The device of claim 10, wherein the stimulation therapy is provided between a first elongate electrode of the plurality of elongate electrodes and an opposite elongate electrode of the plurality of elongate electrodes.

14. A device for delivering thermal or non-thermal ablation, the device comprising:
a sheath;
a handle at a proximal end portion of the sheath;
a balloon at a distal end portion of the sheath; and
a plurality of elongate electrodes surrounding the balloon.

15. (canceled)

16. The device of claim 14, wherein the plurality of elongate electrodes comprises six elongate electrodes, each elongate electrode having a length of about 3 cm to about 7 cm.

17. The device of claim 16, wherein the six elongate electrodes create a hexagonal shape around the balloon.

18. The device of claim 14, wherein the plurality of elongate electrodes are configured to expand and collapse as the balloon inflates and deflates.

19. The device of claim 18, wherein the balloon is configured to inflate to a plurality of expanded configurations with different diameters and wherein the plurality of expanded configurations differ by about 1 mm.

20. (canceled)

21. The device of claim 14, wherein the handle includes a plurality of wire ends of the plurality of elongate electrodes, and wherein the plurality of wire ends are configured to connect to a stimulation device.

22. The device of claim 21, wherein the plurality of elongate electrodes are configured to deliver a stimulation therapy from the stimulation device.

23. The device of claim 22, wherein the stimulation therapy is provided between a first elongate electrode of the plurality of elongate electrodes and an adjacent elongate electrode of the plurality of elongate electrodes.

24. A method of treating or preventing obesity in a subject in need thereof, the method comprising:
providing the device of claim 1; and
delivering a stimulation therapy, generated by the device, to a gastric mucosa of the subject.

25.-35. (canceled)
